(19) United States
(12) Patent Application Publication
(10) Pub. No.: US 2012/0095305 A1
(43) Pub. Date: Apr. 19, 2012

(54) Spectrophotometric Monitoring Of Multiple Layer Tissue Structures

(75) Inventors: Ming Wang, Cupertino, CA (US); Shih-Ping Wang, Los Altos, CA (US); Zengpin Yu, Palo Alto, CA (US); Wei Zhang, Union City, CA (US)

(73) Assignee: O2 MEDTECH, INC., Los Altos, CA (US)

(21) Appl. No.: 12/815,696

(22) Filed: Jun. 15, 2010

Related U.S. Application Data
(60) Provisional application No. 61/187,222, filed on Jun. 15, 2009.

(51) Int. Cl. A61B 5/1455; A61B 6/00

(52) U.S. Cl. 600/323; 600/476

(57) ABSTRACT

Methods, systems, and related computer program products for the non-invasive spectrophotometric monitoring of a biological volume having multiple tissue layers are described. Aggregate absorption and scattering properties are measured for each of a plurality of predetermined source-detector separation distances along a surface of the biological volume, the measurement being based on a model of the biological volume as a single-layer, semi-infinite, homogeneous volume. A predetermined multi-layer tissue model is retrieved that characterizes a mathematical relationship among (a) absorption and scattering properties of each layer of a multi-layer tissue structure, and (b) aggregate absorption and scattering properties of the multi-layer tissue structure as would be measured at selected source-detector separation distances along a surface thereof. The measured aggregate absorption and scattering properties are processed in conjunction with the predetermined multi-layer tissue model to compute therefrom a deep-layer-specific absorption property corresponding to the relatively deep tissue layer.

### CROSS-REFERENCE TO RELATED APPLICATIONS

This patent application claims the benefit of U.S. Provisional Ser. No. 61/187,222, filed Jun. 15, 2009, which is incorporated by reference herein.

### FIELD

This patent specification relates to the non-invasive monitoring of a physiological condition of a patient using information from near-infrared (NIR) optical scans. More particularly, this patent specification relates to systems, methods, and related computer program products for the non-invasive monitoring of relatively deep tissue layers of multiple layer tissue structures, such as may be advantageously employed for non-invasive monitoring of oxygenation levels in the human brain.

### BACKGROUND AND SUMMARY

The use of near-infrared (NIR) light as a basis for the measurement of biological properties or conditions in living tissue is particularly appealing because of its relative safety as compared, for example, to the use of ionizing radiation. Various techniques have been proposed for non-invasive NIR spectroscopy or NIR spectrophotometry (NIRS) of biological tissue. Generally speaking, these techniques are directed to detecting the concentrations of one or more chromophores in the biological tissue, such as blood hemoglobin in oxygenated (HbO) and deoxygenated (Hb) states.

As used herein, NIR tissue oxygenation level monitoring refers to the introduction of NIR radiation (e.g., in the 500-2000 nm range) into a tissue volume and the processing of received NIR radiation migrating outward from the tissue volume to generate at least one metric indicative of oxygenation level(s) in the tissue. One example of an oxygenation level metric is oxygen saturation [SO₂], which refers to the fraction or percentage of total hemoglobin [HbT] that is oxygenated hemoglobin [HbO]. NIRS-based oxygen saturation readings can be classified as “relative” in nature (i.e., presented only in terms of their change over time) or can be “absolute” in nature (i.e., computed from absolute concentrations of [HbO] and [HbT] in units of grams per deciliter (g/dl) or equivalent).

NIR cerebral oxygenation level monitoring, which refers to the transcranial introduction of NIR radiation into the intracranial compartment and the processing of received NIR radiation migrating outward therefrom to generate at least one metric indicative of oxygenation level(s) in the brain, represents one particularly important type NIR tissue oxygenation level monitoring. One exemplary need for reliable determination of oxygen saturation levels in the human brain arises in the context of the millions of surgical procedures performed under general anesthesia every year. One statistic recited in U.S. Pat. No. 5,902,235 is that at least 2,000 patients die each year in the United States alone due to anesthetic accidents, while numerous other such incidents result in at least some amount of brain damage. Certain surgical procedures, particularly of a neurological, cardiac or vascular nature, may require induced low blood flow or pressure conditions, which inevitably involves the potential of insufficient oxygen delivery to the brain. Many surgical procedures also involve the possibility that a blood clot or other clottable material can break free, or otherwise get introduced into the bloodstream, and travel to the brain to cause a localized or widespread ischemic event therein. At the same time, the brain is highly intolerant to oxygen deprivation, and brain cells will die (become infarcted) within a few minutes if not sufficiently oxygenated. Accordingly, the availability of immediate, accurate and reliable information concerning brain oxygenation levels is of critical importance to anesthesiologists and surgeons, as well as other involved medical practitioners.

Pulse oximetry, in which infrared sources and detectors are placed across a thin part of the patient's anatomy such as a fingertip or earlobe, has arisen as a standard of care for all operating room procedures. However, pulse oximetry provides only a general measure of blood oxygenation as represented by the blood passing by the fingertip or earlobe sensor, and does not provide a measure of oxygen levels in vital organs such as the brain. In this sense, the surgeons in the operating room essentially “fly blind” with respect to brain oxygenation levels, which can be a major source of risk for patients (e.g., stroke) as well as a major source of cost and liability issues for hospitals and medical insurers.

Valid NIR cerebral oxygenation level readings provide crucial monitoring data for the surgeon and other attending medical personnel, providing more direct data on brain oxygenation levels than pulse oximeters while being just as safe and non-invasive as pulse oximeters. Generally speaking, such systems involve the attachment of an NIR probe patch, or multiple such NIR probe patches, to the forehead and/or other available skin surface of the head. Each NIR probe patch usually comprises one or more NIR optical source ports for introducing NIR radiation into the cerebral tissue and one or more NIR optical receiver ports for detecting NIR radiation that has migrated through at least a portion of the cerebral tissue. One or more oxygenation level metrics are then provided on a viewable display in a digital readout and/or graphical format.

One issue that arises by virtue of the anatomy of the human head relates to the substantial amount of “non-interesting” or “non-vital” tissue, primarily the skin, skull, and cerebrospinal fluid layers, through which the NIR radiation must pass on its way to and from the brain tissue that is the “interesting” tissue. It would be desirable to provide an NIR-based cerebral oximeter that is designed, calibrated, and subsequently operated in a manner that effectively accounts and/or compensates for the presence and/or characteristics of such “non-interesting” or “non-vital” tissue in the optical path between the NIR probe patch and the “interesting” tissue. Other issues arise as would be apparent to one skilled in the art upon reading the present disclosure.

It is to be appreciated that although one or more preferred embodiments is detailed hereinbelow in the particular context of NIR cerebral oxygenation level monitoring (NIR cerebral oximetry), the present teachings are readily applicable to the non-invasive spectrophotometric monitoring of any of a variety of different body parts in which multiple tissue layers are presented including, but not limited to, the kidney, lung, liver, arm, leg, neck, etc., and furthermore are applicable for the monitoring of any of a variety of different chromophore types therein.

Provided according to one or more preferred embodiments are methods, systems, and related computer program products for the non-invasive spectrophotometric monitoring of a biological volume having multiple tissue layers including a relatively deep tissue layer. At least one measured absorption property and at least one measured scattering property are received for each of a plurality of predetermined source-detector separation distances of an aggregrate near-infrared spectrophotometric (NIRS) tissue monitor as applied along a surface of the biological volume. Preferably, these aggregate NIRS tissue measurements are based on a model of the biological volume as a single-layer, semi-infinite, homogeneous volume. A predetermined multi-layer tissue model is retrieved that characterizes a mathematical relationship among (a) absorption and scattering properties of each layer of a multi-layer tissue structure, and (b) aggregate absorption and scattering properties of the multi-layer tissue structure as would be measured by the aggregate NIRS tissue monitor at selected source-detector separation distances along a surface thereof. The measured absorption and scattering properties of the biological volume for the plurality of predetermined source-detector separation distances is processed in conjunction with the predetermined multi-layer tissue model to compute therefrom a deep-layer-specific absorption property corresponding to the relatively deep tissue layer.

Provided according to one preferred embodiment is an NIR spectrophotometer capable of deep-layer-specific monitoring of a deep layer in a multilayer tissue sample. By deep-layer-specific monitoring, it is meant that an optical property (such as absorption property or scattering property) of a deep layer and/or a particular chromophore level present in a deep layer is monitored separably and distinguishably from the optical property and/or chromophore levels of the other tissue layers in the multilayer tissue sample. For many clinically important situations, deep-layer-specific monitoring represents a preferable advance over what is termed herein “aggregate” tissue monitoring, in which the measured chromophore levels are applicable to the tissue sample as a whole, including the effects of both the non-interesting tissue layers lying between the NIR probe patch and the interesting, vital tissue of interest. In the case of cerebral oxygenation monitoring, it is the brain tissue layer oxygenation levels that are of critical importance, whereas the intervening tissue layers comprising the skin, the skull, and the cerebrospinal (CSF) fluid layers are substantially less important. The effects of those less-important layers can reduce the usefulness and clinical applicability of the readings provided by “aggregate” cerebral oxygenation monitors of the known prior art. Provided according to a preferred embodiment is an NIR spectrophotometer capable of brain-layer-specific oxygenation monitoring, such that errors or variations due to the non-vital intervening tissue layers are obviated and/or inhibited. Although practical NIR spectrophotometric systems have always relied on some combination of first-principles diffusion mathematics and empirical laboratory measurements, one or more of the preferred embodiments described further hereinbelow represents an appreciable shift toward laboratory empiricism in the development and perfection of an NIR cerebral oximeter.

Provided according to one preferred embodiment is a deep-layer-specific NIR cerebral oximeter that is built upon, and incorporates, all or part of one or more known aggregate NIR tissue monitors capable of providing aggregate tissue property measurements. Using an array of sources and detectors positioned over the multilayer tissue sample at multiple predetermined source-detector separation distances, aggregate tissue property measurements are acquired for each source-detector separation distance. In accordance with their aggregate character, the aggregate tissue property measurements (for example, absorption property or absorption coefficient μₐ and scattering property or effective scattering coefficient μₛ) are provided under one or more simplifying assumptions, such as an assumption that the tissue sample has spatially homogenous tissue properties and is semi-infinite in geometry. This semi-infinite, homogenous assumption is referenced as “SI-H” hereinbelow, and corresponding tissue property measurements are referenced as SI-H measurements. The aggregate tissue property measurements for the multiple source-detector separation distances are then processed according to a predetermined, pre-calibrated algorithm to compute deep-layer-specific optical property and/or chromophore level readings for the multi-layer tissue sample, wherein the predetermined, pre-calibrated algorithm has been provided using the steps of (i) acquiring a population of physical (or virtual) multi-layer tissue phantoms having known layer thicknesses and known absorption and scattering properties in each layer, (ii) acquiring a large dataset of aggregate physical measurements (or simulation results) based on those tissue phantoms for the multiple source-detector separation distances, and then (iii) using that large dataset of empirical measurements to compute calibration parameters for a predetermined multi-layer tissue model characterizing a mathematical relationship among the absorption and scattering properties of each layer of a tissue phantom, the thicknesses of each layer of the tissue phantom, and the aggregate absorption and scattering properties of the tissue phantom for each source-detector separation distance.

According to another preferred embodiment, a deep-layer-specific NIR tissue monitor is provided that comprises an array of sources and detectors positioned over the multilayer tissue sample at multiple predetermined source-detector separation distances. Measurements corresponding to the multiple source-detector separation distances are then provided as inputs to a pre-populated lookup table, the lookup table outputting deep-layer-specific optical property and/or chromophore level readings corresponding to the multi-layer tissue sample, wherein the lookup table has been pre-populated using the steps of (i) acquiring a population of physical (or virtual) multi-layer tissue phantoms having known layer thicknesses and known absorption and scattering properties in each layer, (ii) acquiring a large dataset of physical measurements (or simulation results) based on those tissue phantoms for the multiple source-detector separation distances, and then (iii) using that large dataset o physical measurements (or simulation results), known layer thicknesses, known source-detector separation distances, and known layer absorption and scattering properties to populate the lookup table.

According to another preferred embodiment, a deep-layer-specific NIR tissue monitor is provided that comprises an array of sources and detectors positioned over the multilayer tissue sample at multiple predetermined source-detector separation distances. Measurements corresponding to the multiple source-detector separation distances are then provided as inputs to a pre-populated lookup table (or to a predetermined, pre-calibrated algorithm) to result in deep-layer-specific optical property and/or chromophore level readings corresponding to the multi-layer tissue sample, wherein the lookup table has been pre-populated (or the predetermined, pre-calibrated algorithm has been provided) using the steps of (i) non-invasively acquiring reasonable clinical estimations of deep layer tissue properties for a population of human test subjects having variations in deep layer tissue oxygenation levels, (ii) acquiring simultaneously with those reasonable clinical estimations a large dataset of physical measurements on that population of human subjects for the multiple source-detector separation distances, and then (iii) using that large dataset of physical measurements, known layer thicknesses, known source-detector separation distances, and known layer absorption and scattering properties to populate the lookup table.

### BRIEF DESCRIPTION OF THE DRAWINGS

FIG. 1 illustrates deep-layer-specific spectrophotometric monitoring according to a preferred embodiment;

FIG. 2A illustrates calibrating a multi-layer tissue model of a deep-layer-specific spectrophotometric monitoring system according to a preferred embodiment;

FIG, 2B illustrates calibrating a multi-layer tissue model of a deep-layer-specific spectrophotometric monitoring system according to a preferred embodiment;

FIG, 3 illustrates a cross-sectional view of a probe patch of an aggregate near-infrared spectrophotometric (NIRS) monitor having multiple source-detector separation distances as applied to a multi-layer tissue sample according to a preferred embodiment;

FIG. 4 illustrates a multi-layer tissue model according to a preferred embodiment;

FIG. 5 illustrates calibrating a multi-layer tissue model according to a preferred embodiment;

FIG. 6 illustrates deep-layer-specific spectrophotometric monitoring according to a preferred embodiment;

FIG. 7A illustrates an aggregate NIRS tissue monitor of a deep-layer-specific spectrophotometric monitoring system according to a preferred embodiment;

FIG. 7B illustrates two-layer tissue phantoms associated with a two-layer tissue model according to a preferred embodiment;

FIG. 7C illustrates a cross-sectional view of a probe patch of an aggregate NIRS monitor having two source-detector separation distances as applied to a two-layer tissue sample according to a preferred embodiment;

FIG. 7D illustrates a two-layer tissue model according to a preferred embodiment;

FIG. 8 illustrates calibrating the two-layer tissue model of FIG. 7D according to a preferred embodiment;

FIG. 9 illustrates deep-layer-specific spectrophotometric monitoring according to a preferred embodiment;

FIG. 10A illustrates a cross-sectional view of a probe patch of an aggregate NIRS monitor having three source-detector separation distances as applied to a three-layer tissue sample according to a preferred embodiment;

FIG. 10B illustrates a three-layer tissue model according to a preferred embodiment;

FIGS. 11A-11D illustrate alternative two-layer tissue models corresponding to thin, average, and thick top-layer thicknesses, respectively, according to a preferred embodiment;

FIG. 12 illustrates deep-layer-specific spectrophotometric monitoring according to a preferred embodiment;

FIG. 13 illustrates calibrating a multi-layer tissue model according to a preferred embodiment;

FIG. 14 illustrates deep-layer-specific spectrophotometric monitoring according to a preferred embodiment;

FIG. 15 illustrates calibrating a multi-layer tissue model according to a preferred embodiment;

FIG. 16 illustrates deep-layer-specific spectrophotometric monitoring according to a preferred embodiment;

FIGS. 17A-17C illustrate calibrating a multi-layer tissue model according to a preferred embodiment;

FIG. 18 illustrates deep-layer-specific spectrophotometric monitoring according to a preferred embodiment; and

FIG. 19 illustrates calibrating a multi-layer tissue model according to a preferred embodiment.

### DETAILED DESCRIPTION

As used herein, the terms “top layer(s),” “outer layer(s)”, and “surface layer(s)”, are generally used to refer to the one or more “non-interesting” or “non-vital” tissue layers that intervene between the NIR probe patch and the “interesting” tissue. As used herein, the term “deep layer” refers to the “interesting” tissue that is disposed beneath the top/outer/surface tissue layer(s) relative to the NIR probe patch. For two-layer tissue models, the “deep layer” may also be referenced as the “bottom layer.” However, it is to be appreciated that the term “deep layer,” as used herein, does not necessarily refer to the anatomically deepest part of the body part under study relative to the skin surface. For example, in renal oxygenation level monitoring, the kidney represents the “deep layer” as that term is used herein, even though there are other internal tissues that lie anatomically deeper than the kidney relative to the skin surface. Due to the high signal losses generally associated with NIR spectrophotometry, any tissues that are deeper than the interesting “deep layer” can often be ignored and/or considered as being integral with the “deep layer.” Accordingly, in the examples hereinbelow, the “deep layer” containing the “interesting” tissue corresponds to the bottom-most layer (N^{th} layer) of the particular N-layer tissue model being described. However, it is to be appreciated that the scope of the preferred embodiments is not so limited, with the present teachings readily encompassing scenarios in which the “deep layer” of interesting tissue is actually an m^{th} layer, 1<m<N, of the particular N-layer tissue model being described.

The preferred embodiments set forth further herein are preferably implemented using phase modulation spectrophotometry (PMS) systems for the aggregate readings. PMS-based systems, which are sometimes termed intensity modulation spectroscopy systems and sometimes termed frequency domain spectroscopy systems, are known in the art and are discussed, for example, in U.S. Pat. No. 4,972,331, U.S. Pat. No. 5,187,672, U.S. Pat. No. 5,492,118, U.S. Pat. No. 5,497,769, and WO1994/21173A1, each of which is incorporated by reference herein. It is to be appreciated, however, that the present teachings could be readily adapted for use in conjunction with continuous wave spectrophotometry (CWS) systems, which are discussed in WO1992/20273A2 and WO1996/16592A1, each of which is incorporated by reference herein. It is to be further appreciated that the present teachings could be readily adapted for use in conjunction with time resolved spectrophotometry (TRS) systems, which are discussed in U.S. Pat. No. 5,119,815, U.S. Pat. No. 5,386,827, and WO1994/22361A1, each of which is incorporated by reference herein.

Preferably, for any particular source-detector separation distance relevant to an aggregate PMS measurement, a balanced arrangement of sources and detectors is used, for example, two sources S1 and S2 and two detectors D1 and D2 place at the corners of a rectangle such that the S1-D1 distance is equal to the S2-D2 distance, and such that the S1-D2 distance is equal to the S2-D1 distance, thereby allowing, using known methods, the aggregate PMS readings to be reliably obtained in a manner that is generally independent of the coupling efficiency between the skin and each different source or detector. Each of the following references is incorporated by reference herein: U.S. Pat. No. 6,078,833, U.S. Pat. No. 6,615,061B1, U.S. Pat. No. 6,574,490B2, U.S. Pat. No. 6,630,673B2, U.S. Pat. No. 6,766,188B2, U.S. Pat. No. 7,072,701B2, U.S. Pat. No. 7,532,919B2, and WO2008117286A2. For notational simplicity hereinbelow, the symbol μₛ is used to represent the effective scattering coefficient, sometimes referenced as the reduced scattering coefficient or scattering property, even though that quantity, which is related to the scattering coefficient by a straightforward relationship involving the average scattering angle, is often referenced in the literature as μ′ₛ.

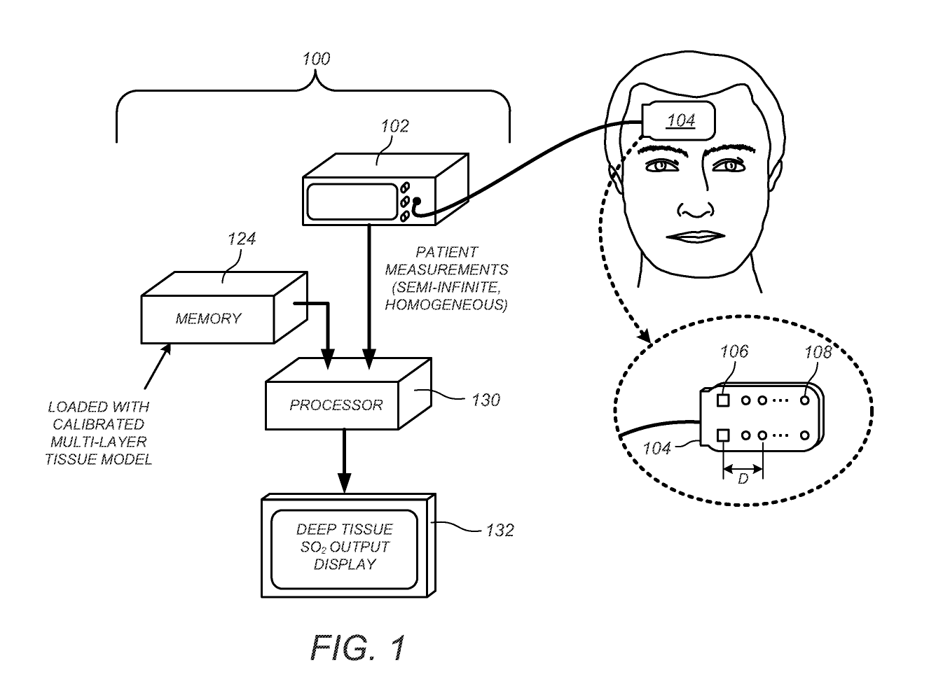

FIG. 1 illustrates a deep-layer-specific NIR cerebral oximeter 100 according to a preferred embodiment, comprising an aggregate NIR oximeter 102, a probe patch 104, a memory 124, a processor 130, and an output display 132. For any particular source-detector separation distance, the aggregate NIR oximeter 102 is capable of measuring an aggregate absorption coefficient and aggregate scattering property as if the tissue sample were a homogeneous, semi-infinite medium (SI-H), for each of a plurality of wavelengths such as 690 nm and 830 nm, at each of a plurality different distances D between sources 106 and detectors 108 of the probe patch 104. Systems and methods for measuring an aggregate absorption coefficient and aggregate scattering property as if the tissue sample were a homogeneous, semi-infinite medium (SI-H) would be apparent to a person skilled in the art in view of the present disclosure, with one exemplary discussion of such methods being provided in Fantini, et. al., “Semi-Infinite-Geometry Boundary Problem for Light Migration in Highly Scattering Media: A Frequency Domain Study in the Diffusion Approximation,” J. Opt. Soc. Am. B. Vol. 11, No. 10, pp. 2128-2138 (October 1994), which is incorporated by reference herein.

By way of example and not by way of limitation, in one preferred embodiment there may be four (4) discrete source-detector separation distances of 1.5 cm, 3 cm, 4.5 cm, and 6 cm, respectively. For this case, and for the preferred balanced arrangement described in the preceding paragraph, there are thus two (2) sources 106 and eight (detectors) 108 on the probe patch 104. In another preferred embodiment, the source-detector separation distances may be 2 cm, 4 cm, 6 cm, and 8 cm, respectively. In a simplest preferred embodiment, there may be only two (2) distinct source-detector separation distances D, such as 2 cm and 6 cm. In more complex preferred embodiments, there may be eight (8), then (10), or even twelve (12) or more distinct source-detector separation distances D.

Memory 124 stores a pre-calibrated, predetermined multi-layer tissue model characterizing a mathematical relationship among the absorption and scattering properties of each layer of a tissue structure, the thicknesses of each layer of the tissue structure, and the aggregate absorption and scattering properties of the tissue structure for each source-detector separation distance. Processor 130 is programmed and configured to execute a predetermined, pre-calibrated algorithm based on the pre-calibrated, predetermined multi-layer tissue model and the aggregate absorption coefficient and aggregate scattering properties to generate a deep-tissue-specific SO₂ reading for the patient, which is output onto the display 132. For one preferred embodiment, the aggregate absorption and scattering properties are processed in conjunction with the pre-calibrated, predetermined multi-layer tissue model to compute a deep-layer-specific absorption property μ_{aDEEP}, and then a deep-tissue-specific reading SO_{2,DEEP} is derived from the deep-layer-specific absorption property μ_{aDEEP}. By way of example, deep-layer-specific absorption coefficients μ_{aDEEP}(690) and μ_{aDEEP}(830) can be computed for respective NIRS wavelengths of 690 nm and 830 nm, and then the deep-tissue-specific reading SO_{2,DEEP} can be computed using the formula SO_{2,DEEP}=1.0941-0.4284{μ_{aDEEP}(690)/μ_{aDEEP}(830)}.

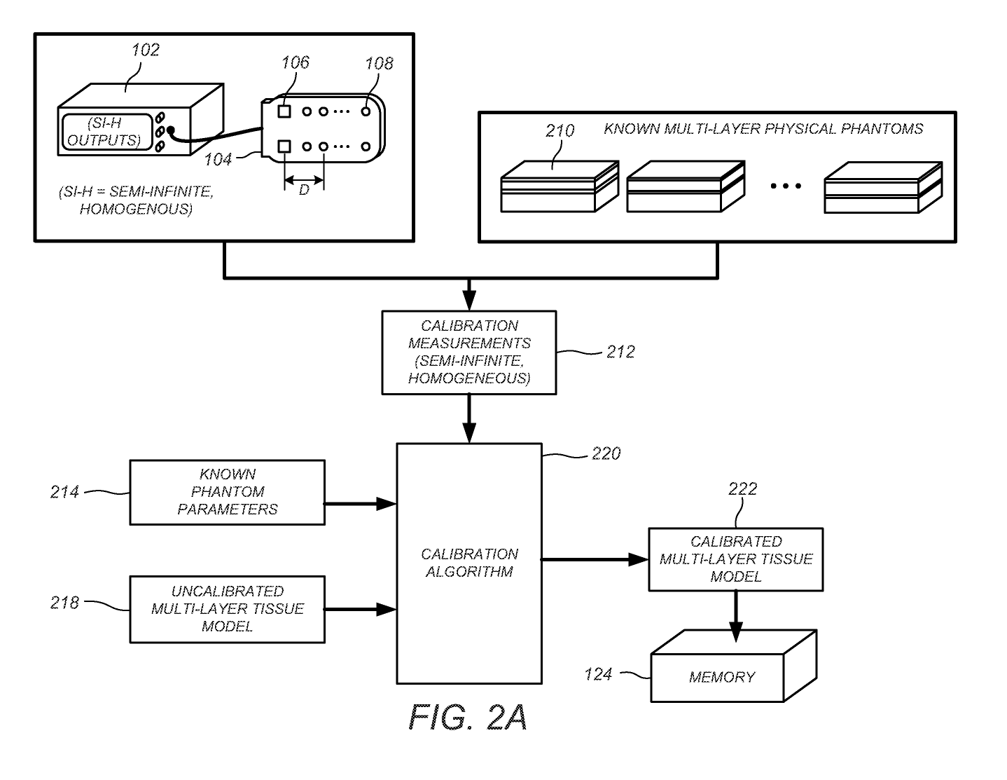

FIG. 2A illustrates calibrating a deep-layer-specific NIR cerebral oximeter according to a preferred embodiment. A relatively large population of multi-layer head phantoms 210 is provided, with each layer of each head phantom having a known thickness, a known absorption coefficient, and a known scattering property. Any of a variety of known methods (e.g., off-the-shelf solid materials, liquid solutions containing different levels of ink pigment, lipid materials, etc.) can be used to physically realize the laboratory population of head phantoms.

Each head phantom comprises two or more layers, with the total number of layers “L” being preselected according to the dimensional extent of the chosen multi-layer tissue model. Choosing a larger number of layers “L” can lead to a multilayer model that more closely approximates the structure of the multi-layer tissue sample than choosing a fewer number of layers, provided that the enlarged dimensional extent of the calibration computations and clinical usage computations can be accommodated. Choosing a lesser number of layers provides for easier modeling and computation. Either choice (larger number for “L”, lesser number for “L”) is within the scope of the present teachings. The absorption and scattering properties of any particular layer will be chosen to vary systematically over expected ranges thereof across the phantoms. Also, the many head phantoms 210 will include many different combinations and permutations of those values across the different layers. In the general case, the many head phantoms can also have variations in the thicknesses of different layers, representing the fact that different people can have different skin thicknesses, different skull thicknesses, and so on. Alternatively, to keep the dimensionality and complexity of the mathematical calibration problem within certain limits, the phantoms 210 can all have the same fixed, predetermined layer thickness profile that represents a best clinical estimate of the layer thickness profiles for the actual patients to be monitored.

For one preferred embodiment, the head phantoms 210 can comprise four (4) layers for modeling the skin layer, skull layer, cerebrospinal fluid (CSF) layer, and, finally, the brain layer of patient tissue. Ranges of values of the thicknesses of these layers can be acquired from any of a variety of medical textbooks or other sources. Ranges of values for the absorption coefficients and scattering properties of each layer can likewise be acquired from any of a variety of textbooks or other sources. One example of the latter is Vo-Dinh, ed., Biomedical Photonics Handbook, CRC Press (2003), which contains tables of in vivo and in vitro measurements of absorption coefficients and scattering properties for various body parts in various conditions collected from a range of information sources.

Returning now to the calibration process of FIG. 2A, a large population of calibration measurements 212 is taken, using aggregate NIR oximeter 102 with probe patch 104, to produce values of an aggregate absorption coefficient μ_{aA} and aggregate scattering property μ_{sA} for each different separation distance D for each head phantom. Then, based on the measurements 212 and the known phantom parameters 214, and the using the framework of a predetermined but uncalibrated multi-layer tissue model 218, a calibration algorithm 220 is performed that computes all of the parameters necessary to create a calibrated multi-layer tissue model 222, which is then stored in the memory 124. The deep-layer-specific NIR cerebral oximeter 100 is then ready for clinical use in the system of FIG. 1, supra.

FIG. 2B illustrates calibrating a deep-layer-specific NIR cerebral oximeter according to another preferred embodiment similar to that of FIG. 2A except that virtual head phantoms 210′ existing only as digital arrays are used in place of physical head phantoms 210, and except that computer simulation results 212′ are computed instead of physical measurements being taken. The computer simulation results are computed to emulate what would be measured by the aggregate NIR oximeter 102, by entering into the simulator the same source powers, source aperture shapes, detector aperture shapes, source-detector separation distances, etc., as are physically presented by the aggregate NIR oximeter 102 that will be used in the deep-layer-specific NIR cerebral oximeter 100. For one preferred embodiment, the computer simulation can use a finite element method in which the behavior of each element is governed by diffusion theory principles for highly scattering media, using an off-the-shelf simulation package such as the COMSOL Multiphysics® software environment available from COMSOL, Inc., of Burlington, Mass.

FIG. 3 illustrates a cross-sectional view of a multi-layer tissue phantom 210 from FIGS. 2A-2B, supra, to which the probe patch 104 is applied. As illustrated in FIG. 3, each separate layer has its own known thickness, known absorption coefficient, and known scattering property. For cerebral oximetry in which the deepest layer (the brain) is the layer of interest, the thickness of the bottom layer “L” can be modeled as very large number or a don't-care parameter.

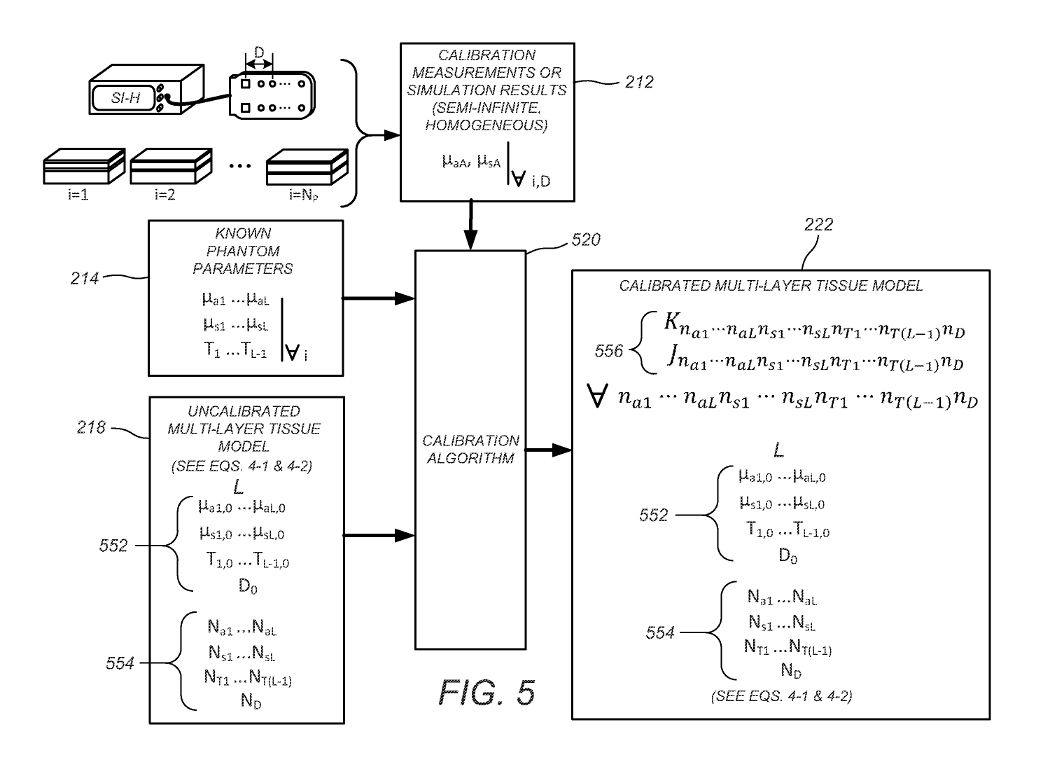

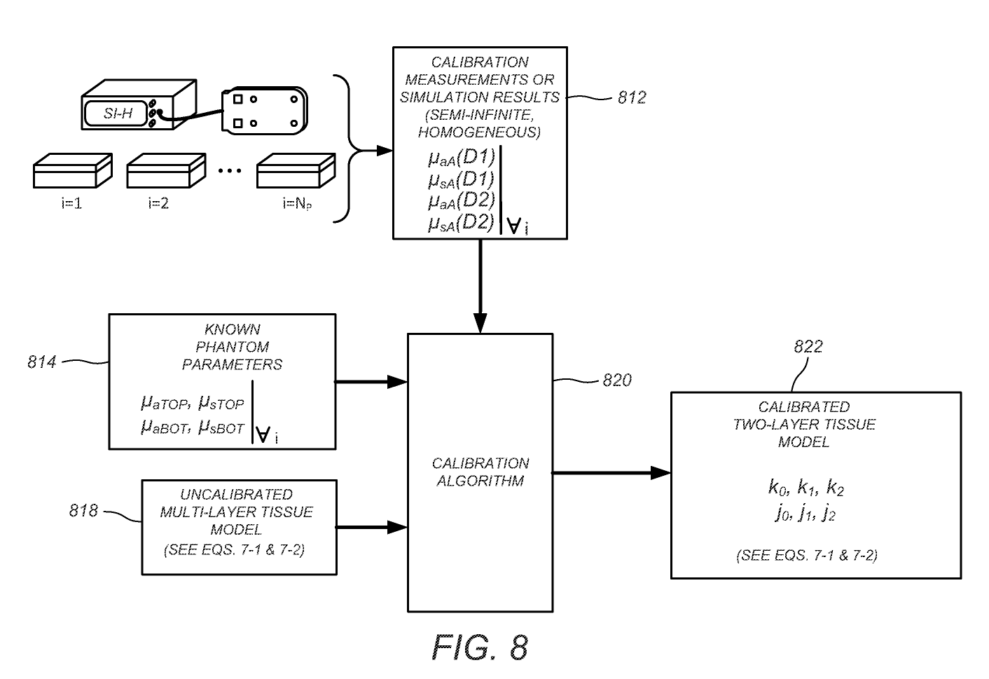

As illustrated by the “banana” shaped “paths” in FIG. 3, certain simplifying assumptions can be made regarding the minimal effect that deeper tissue layers have on aggregate oximetry readings for closely-spaced source-detector pairs. These simplifying assumptions serve as a basis for the preferred embodiments of FIGS. 7C-7D and FIGS. 10A-10B infra, in which calibration computations are made substantially easier by a “layer by layer” approach, in which parameter determinations for each higher (shallower) layer can be used to establish parameter determinations for the next successively lower (deeper) layer. However, the scope of the present teachings does not necessarily require sequential computation of layer properties, as will be appreciated in view of the generalized case next disclosed herein.

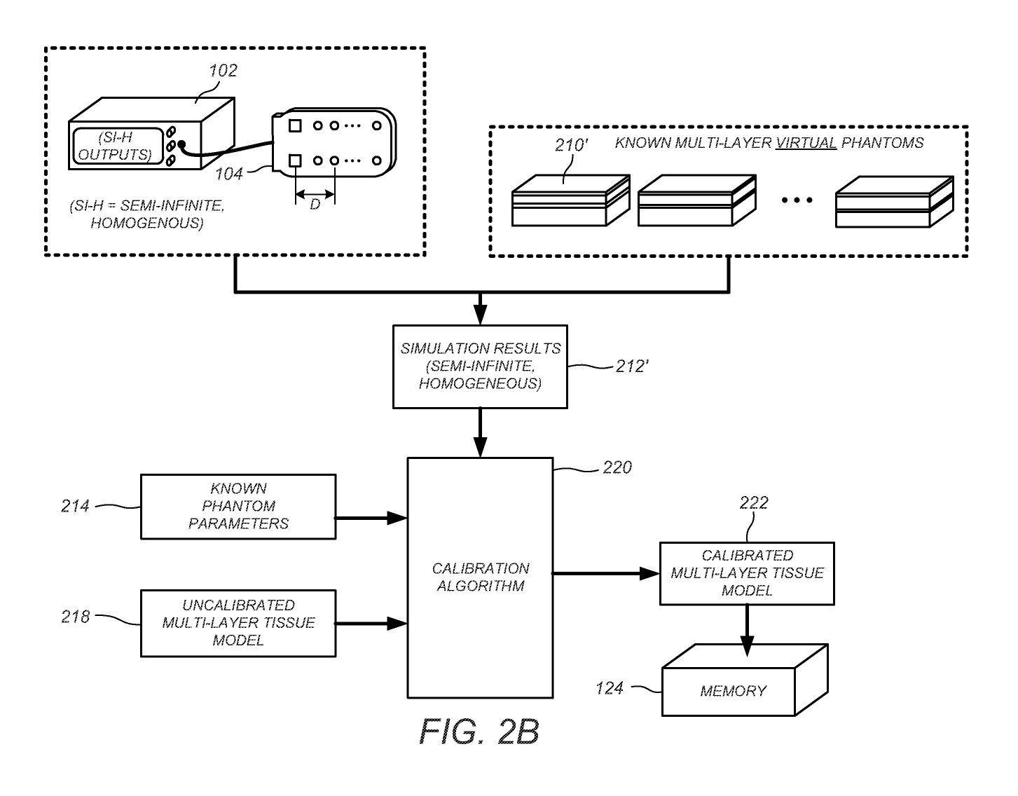

FIG. 4 illustrates an example of a predetermined multi-layer tissue model according to a generalized case of an L-layer tissue model and nonlinear relationships among one or more tissue parameters. The predetermined multi-layer tissue model of FIG. 4 expresses, in Eq. {4-1}, the aggregate absorption coefficient as a variable-order Taylor series expansion of (i) the absorption coefficient of each layer (μₐ₁, . . . , μ_{aL}), (ii) the scattering coefficient of each layer (μₛ₁, . . . , μ_{sL}), (iii) the thickness of each layer above the deepest layer (T₁, . . . , T_{L}), and (iv) the source-detector separation distance D, with Taylor coefficients K_{na1 . . . naLns1 . . . nsLnT1 . . . . nT(L-1)nD}. The predetermined multi-layer tissue model of FIG. 4 similarly expresses, in Eq. {4-2}, the aggregate scattering property as a variable-order Taylor series expansion of those same parameters, with Taylor coefficients J_{na1 . . . naLns1 . . . nsLnT1 . . . nT(L-1)nD}. As used herein, the term “internal model parameters” refers collectively to the Taylor coefficients K_{na1 . . . naLns1 . . . .nsL1 . . . nT(L-1)nD} and J_{na1 . . . naLns1 . . . nsLnT1 . . . nT(L-1)nD}.

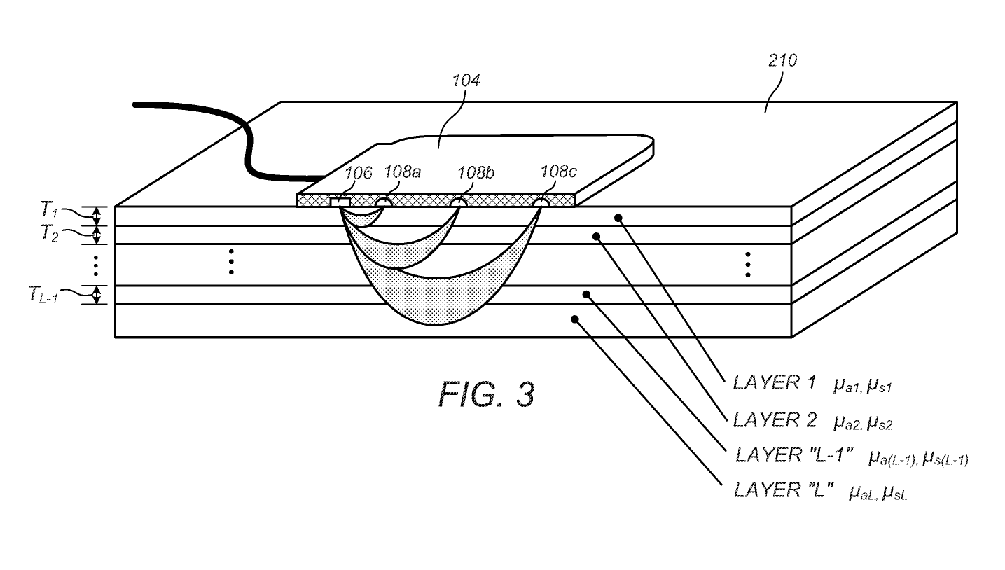

FIG. 5 illustrates the calibration method of FIGS. 2A-2B as applied to the predetermined multi-layer tissue model of FIG. 4 according to a preferred embodiment. Predetermination of the multi-layer tissue model 218 involves the selection of the number of layers “L” in the model, the orders 554 (Nₐ₁, . . . , N_{aL}, Nₛ₁, . . . , N_{sL}, N_{T1}, . . . , N_{T(L-1)}N_{D}) to which each variable will be expanded in the Taylor expansion and (optionally) the base values 552 (μ_{a1,0}, . . . , μ_{aL,0}, μ_{s1,0}, . . . , μ_{sL,0}, T_{1,0}, . . . , T_{(L-1),0},D₀) around which variable is expanded. Prior to the calibration process, it is only these orders 554, base values 552, and number of levels “L” that are known, as well as the phantom properties 214, whereas the internal model parameters are unknown. After the measurement (or simulation) process at step 212, the measured (or simulated) aggregate values μ_{aA} and μ_{sA} become known values for the left sides of Equations {4-1} and {4-2}. The many instances of Equations {4-1} and {4-2} can then be processed by calibration algorithm 520 to compute the internal model parameters 556, at which point the predetermined multi-layer tissue model is now calibrated. Methods for implementing the calibration algorithm 520 would be apparent to a person skilled in the art, including persons skilled in the field of system identification, in view of the instant disclosure without undue experimentation. The internal model parameters 556 may be computed by the calibration algorithm 520 using known methods including, but not limited to, linear regression analysis, non-linear regression analysis, polynomial regression analysis, and partial least-squares algorithms. In other preferred embodiments, a more complex regression model such as a neural network can be used to determine the internal model parameters 556.

The calibration process proceeds separately for each wavelength, and the resultant values for the internal model parameters 556 will be generally be different for different wavelengths. Depending on the particular PMS-based system being used, multiple modulation frequencies may be employed by the aggregate NIR tissue oximeter 102 (e.g., 100 MHz, 200 MHz) in obtaining the aggregate values μ_{aA} and μ_{sA} for that wavelength. However, systems for which the aggregate NIR tissue oximeter 102 only uses a single modulation frequency at any particular wavelength are also within the scope of the present teachings.

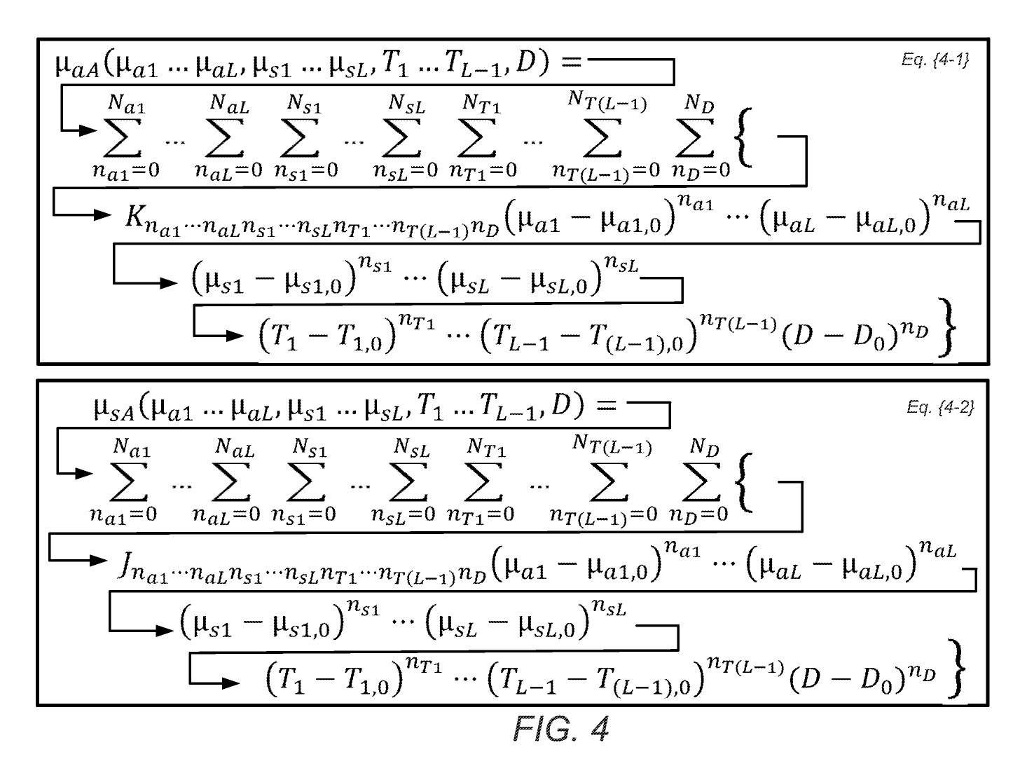

FIG. 6 illustrates a method for computing deep-layer-specific SO₂ readings for an unknown patient using an NIR cerebral oximeter as pre-calibrated according to the method of FIG. 5. After the calibration process of FIG. 5, there exists a calibrated multi-layer tissue model 222 for each wavelength of operation, which are referenced as elements 222(λ₁) and 222(λ₂) in FIG. 6. The calibrated multi-layer tissue model 222(λ₁) comprises the base values 552, the orders 554, the number of layers “L”, and the internal parameters 556 for the wavelength λ₁. The calibrated multi-layer tissue model 222(λ₂) will usually (but is not necessarily required to) comprise the same base values 552, orders 554, and the number of layers “L” as the calibrated multi-layer tissue model 222(λ₁), but will have different internal parameters 556 applicable for the wavelength λ₂. Examples of values for λ₁ and λ₂ are 690 nm and 830 nm, respectively.

In the clinical application process of FIG. 6 on the unknown patient, at step 602 patient measurements are taken to acquire the values μ_{aA} and μ_{sA} for the multiple source-detector separation distances for each wavelength λ₁ and λ₂. For wavelength λ₁, the many instances of Equations {4-1} and {4-2} are solved at step 604 using the calibrated multi-layer tissue model 222(λ₁) to compute the absorption coefficient at layer “12 at wavelength λ₁, designated herein μ_{aL}(λ₁). For wavelength λ₂, the many instances of Equations {4-1} and {4-2} are also solved at step 604 using the calibrated multi-layer tissue model 222(λ₂) to compute μ_{aL}(λ₂). Upon computing μ_{aL}(λ₁) and μ_{aL}(λ₂) for two wavelengths on opposite sides of the isosbestic wavelength for [Hb] and [HbO], a deep-layer-specific blood oxygen saturation reading SO_{2,L} can be readily computed at step 606 known methods and then displayed on output display 232. For example, for the particular case of λ₁ and λ₂ at 690 nm and 830 nm, respectively, the deep-layer-specific SO_{2,L} reading corresponding to the L^{th} layer can be computed as SO_{2,L}=1.0941-0.4284{μ_{aL}(690)/μ_{aL}(830)}.

For the particular preferred embodiment of FIG. 6, as illustrated at step 604 thereof, each of the absorption coefficients μₐ₁, . . . , μ_{a(L-1)}, as well as each of the scattering properties μₛ₁, . . . , μ_{sL} are also computed during the process of computing μ_{aL}. Optionally, those additional results and/or SO₂ readings based thereon can be displayed to the user, which may serve a useful purpose in their own right (e.g., as validation checks). However, depending on the particular mathematical models and assumptions used, those additional results might not be independently observable and therefore would not be available for display. Preferred embodiments in which the additional results are observable independent of μ_{aL}, and preferred embodiments in which the additional results are not observable independent of μ_{aL}, are all within the scope of the present teachings.

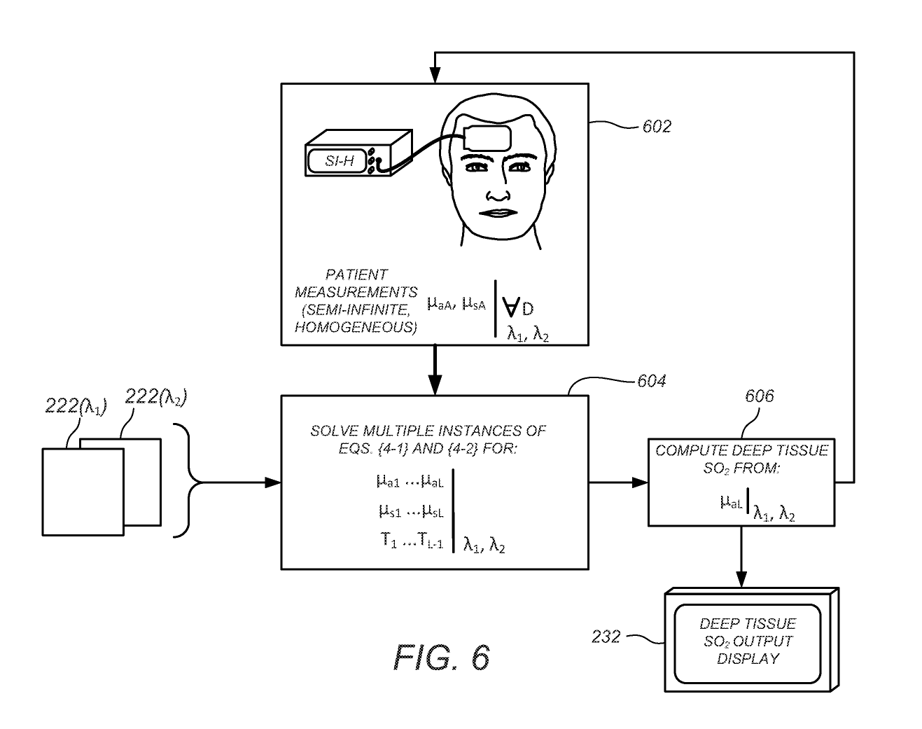

FIGS. 7A-7D, FIG. 8, and FIG. 9 collectively illustrate a version of the preferred embodiments of FIGS. 3-6, supra, with several simplifying assumptions applied. An aggregate NIR oximeter 702 is provided with a probe patch 704 having sources 706 and detectors 708 that define only two (2) distinct source-detector separation distances. The physical (or virtual) tissue phantoms 710 have only two layers each, with the top layers thereof being of a uniform, fixed thickness. Finally, the model is limited to first (linear) order relationships as illustrated in Equations {7-1} and {7-2} of FIG. 7D. Calibrations steps/elements 812, 814, 818, 820, and 822 of FIG. 8 proceed similarly to corresponding steps/elements 212, 214, 218, 220, and 222, respectively, of FIG. 5, supra, with the difference being that the calibration algorithm 820 can proceed in a much simpler, “layer by layer” fashion in which determinations for the top layer measurements (for example, μ_{aA}(D1)=μ_{aTOP}) are used to establish determinations for the bottom layer (for example, causing fewer unknowns in μ_{aA}(D2)=k₀+k₁μ_{aTOP}+k₂μ_{aBOT}), which substantially eases computations. Forward clinical usage steps/elements 822(λ₁), 822(λ₂), 902, 904, 906, and 232 proceed similarly to corresponding steps/elements 222(λ₁), 222(λ₂), 602, 604, 606, and 232, respectively, of FIG. 6, supra, again with the difference that the computations are now substantially easier.

FIGS. 10A-10B illustrate a three-layer tissue phantom and a corresponding three-layer tissue model, respectively, according to a preferred embodiment. Preferably, the number of distinct source-detector distances (three in this example) should be greater than or equal to the number of layers in the multi-layer tissue model (three in this example).

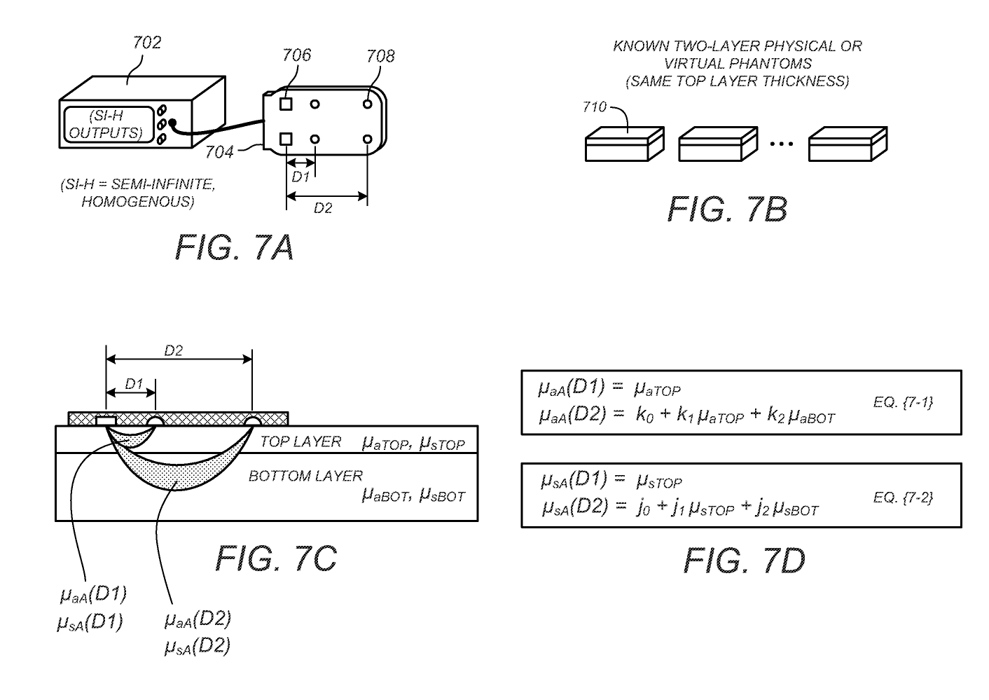

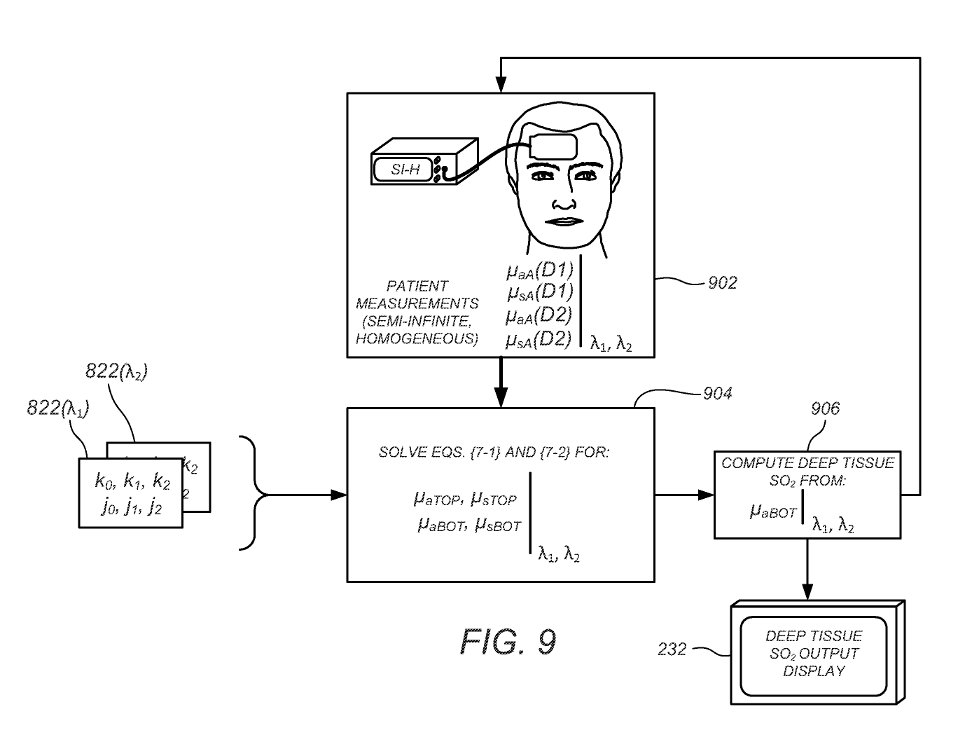

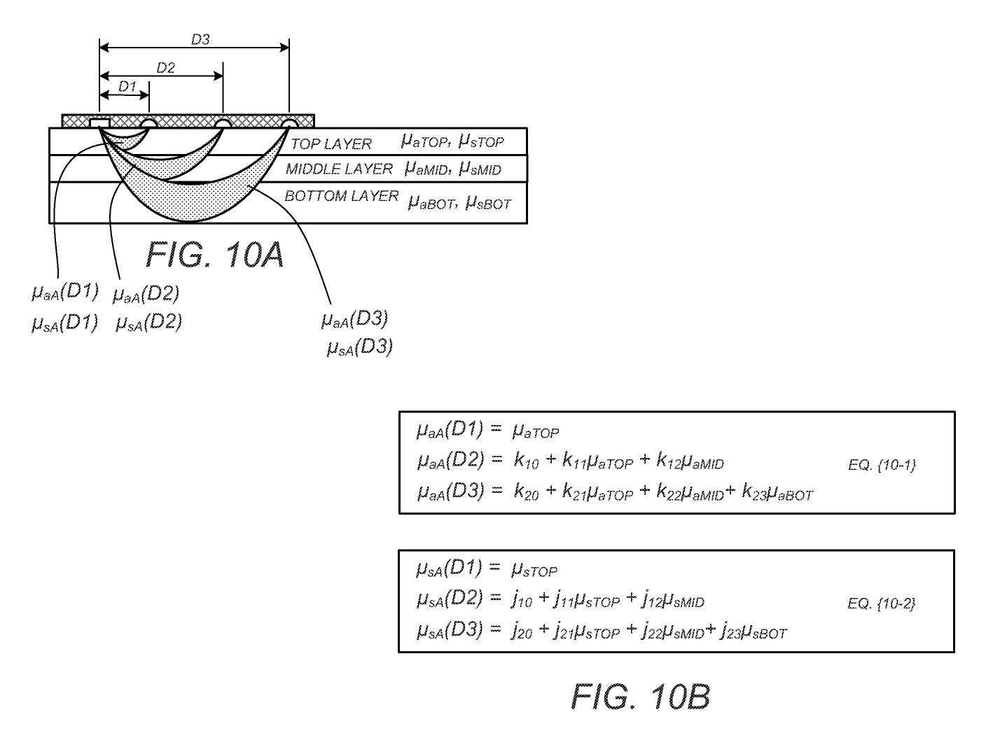

FIGS. 11A-11D, and FIG. 12 illustrate multiple sets of dual-layer tissue phantoms, corresponding versions of dual-layer tissue models, and forward usage of a corresponding deep-tissue-specific NIR cerebral oximeter, respectively, according to a preferred embodiment. In this preferred embodiment, there is provided a certain advantageous amount of increased patient specificity in terms of skull thickness (or, more particularly, the combined thickness of the skin, skull, and CSF modeled as a single top layer), while also retaining a certain advantageous amount of model simplicity, by avoiding the full incorporation of layer thicknesses “T” into the generalized model of FIG. 4, supra. In this preferred embodiment, there are essentially three complete models that are calibrated individually and distinctly from each other, including a first model (FIG. 11B, FIG. 12 block 1222a) for a “thin” skull, a second model (FIG. 11C, FIG. 12 block 1222b) for an “average” skull thickness, and a third model (FIG. 11D, FIG. 12 block 1222c) for a “thick” skull. Measurement, equation solving, and SO₂ calculation steps 1202-1206 proceed in a manner similar to the steps 902-906 of FIG. 9, supra, except that the particular skull model (thin, average, or thick) is dynamically selectable at step 1250, with only one of the models being used at any particular time.

For one preferred embodiment, the results μ_{aTOP} and μ_{aBOT} are monitored on a continuous basis for some period after application of the probe patch to the patient, and the particular model (thin, average, or thick) is chosen based on the behavior of μ_{aTOP}, μ_{aBOT}, μ_{sTOP}, and μ_{sBOT}. For another preferred embodiment (not shown), the particular model (thin, average, or thick) can be chosen by the user (i.e., the attending medical professional) based on externally acquired metrics (e.g., imaging the head with a different imaging modality such as ultrasound or MRI to determine or estimate combined skin, skull, and CSF thickness) and/or based on observed outputs of the system.

For one preferred embodiment, the decision of thin, average, or thick can be made in an iterative process based on expected differential values and/or behaviors of μ_{aTOP}, μ_{aBOT}, μ_{sTOP}, and μ_{sBOT}, and/or SO₂ metrics corresponding thereto, responsive to changes in candidate settings for the top-layer thickness. By way of simplified example, a first “thin” candidate setting is made and the values of μ_{aTOP}, μ_{aBOT}, μ_{sTOP}, and μ_{sBOT} observed. The candidate setting is then switched to “average,” and then to “thick,” with the resultant values of μ_{aTOP}, μ_{aBOT}, μ_{sTOP}, and μ_{sBOT} observed at each setting. Generally speaking, it is known clinically for in vivo measurements that the skull has a higher μₐ range (μₐ about 0.30-0.50 cm⁻¹) than both brain gray matter (μₐ about 0.18-0.25 cm⁻¹) and brain white matter (μₐ about 0.05-0.10 cm⁻¹). It is likewise known clinically for in vivo measurements that the skull has a lower μₛ range (μₛ about 8.5-14.0 cm⁻¹) than both brain gray matter (μₛ about 20-25 cm⁻¹) and brain white matter (μₛ about 40-60 cm⁻¹). If there are substantial changes in the top-layer readings in the form of a μ_{aTOP} decrease and a μ_{sTOP} increase upon changing of the thickness setting from “thin” to “average,” then the “thin” setting should be selected for this patient, because the change to “average” has evidently caused some of the brain tissue to be included in the top-layer reading, meaning that the skull is likely closer to “thin” than to “average” in thickness. If there are not substantial changes in the top-layer readings in the form of a μ_{aTOP} decrease and a μ_{sTOP} increase upon changing of the top-layer thickness setting from “thin” to “average,” then the skull will belong in either the “average” category or “thick” category. In deciding between the “average” category versus the “thick” category, if there are substantial changes in the top-layer readings in the form of a μ_{aTOP} decrease and a μ_{sTOP} increase upon changing of the top-layer thickness setting from “average” to “thick,” then the “average” setting should be selected for this patient, because the change to “thick” has evidently caused some of the brain tissue to be included in the top-layer reading, meaning that the skull is more likely “average” than “thick” in thickness. If there are not substantial changes in the top-layer readings in the form of a μ_{aTOP} decrease and a μ_{sTOP} increase upon changing of the top-layer thickness setting from “average” to “thick,” then the skull is placed in the “thick” category, because the change to “thick” evidently did not cause some of the brain tissue to be included in the top-layer reading. If anomalous readings occur, such as a μ_{aTOP} increase and/or a μ_{sTOP} decrease upon increasing the selected top-layer thickness, then the process can simply be aborted and the “average” thickness category used. Any of a variety of similar practical schemes can be used to select among “thin,” “average,” or “thick” without departing from the scope of the present teachings. Likewise, the thicknesses of each of two or more upper layers can be selected without departing from the scope of the present teachings. Moreover, the number of discrete possible thicknesses for each upper layer(s) can be increased beyond three without departing from the scope of the present teachings.

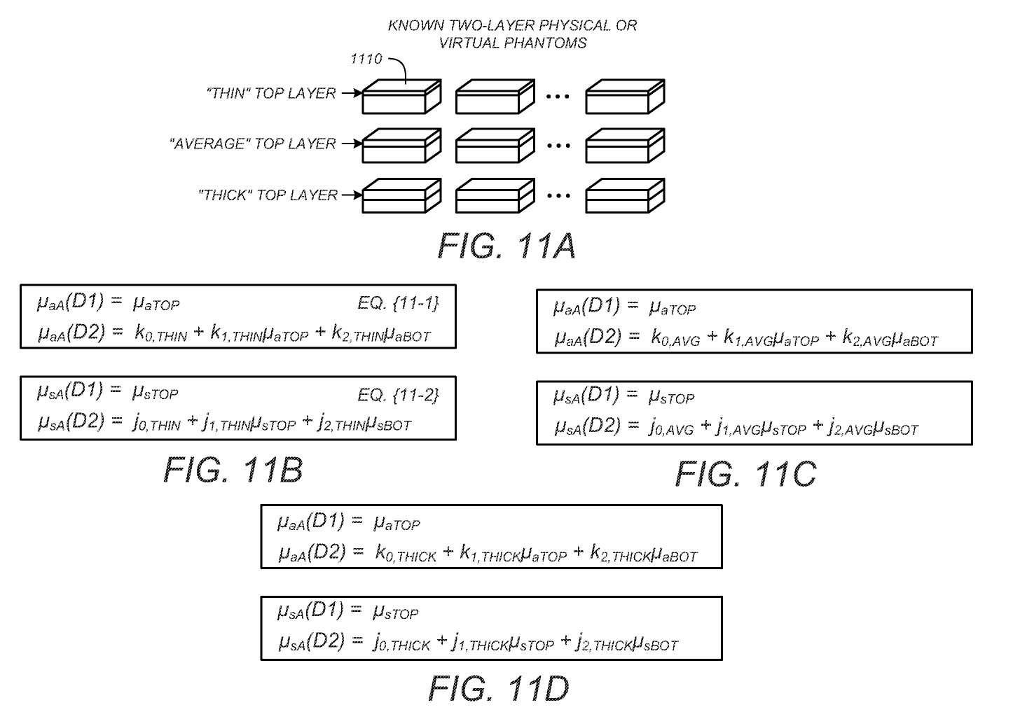

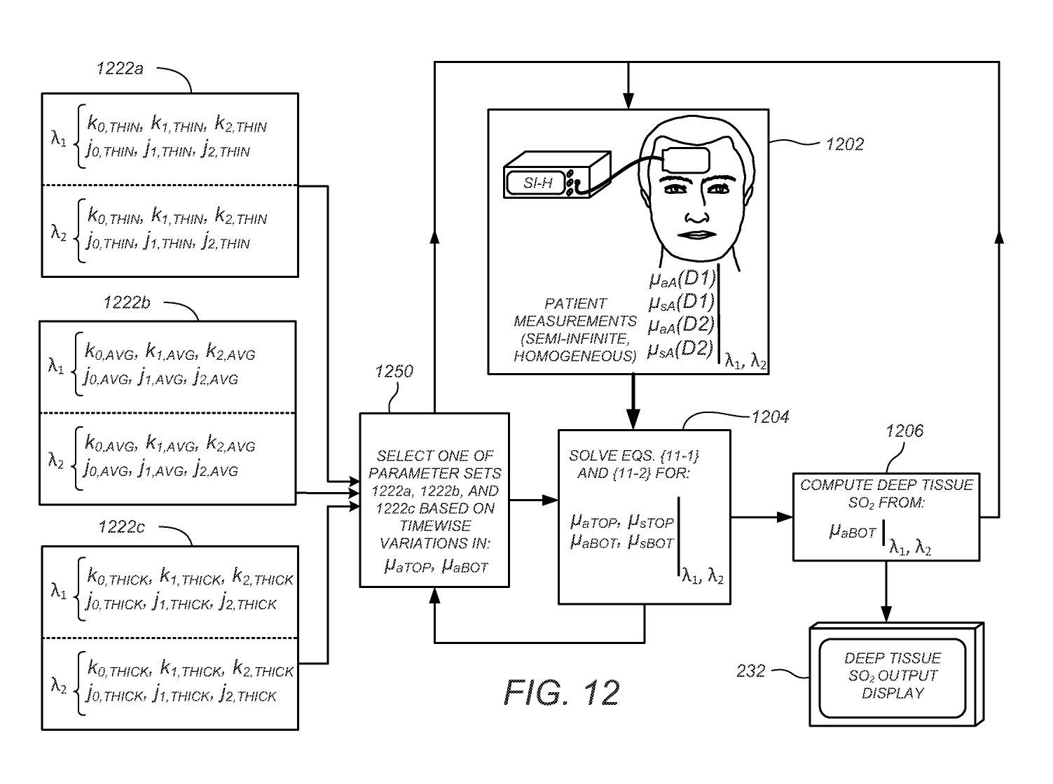

FIGS. 13-14 illustrate calibrating a deep-layer-specific NIR cerebral oximeter, and clinically using the calibrated deep-layer-specific NIR cerebral oximeter, respectively, according to a preferred embodiment in which a lookup table 1370 is used. Although described hereinbelow in terms of dual-layer phantoms and a dual-layer tissue structure assumption, the preferred methods and systems are readily applicable for three or more tissue layers, and such applications are within the scope of the present teachings. Generally speaking, as illustrated in FIG. 14, the purpose of lookup table 1370 is to receive, for each wavelength, SI-H (semi-infinite, homogenous assumption) calibration measurements at multiple source-detector spacing distances from an aggregate NIR tissue oximeter as inputs, and to provide a direct readout 1404 of the deep layer absorption coefficient μ_{aBOT} (or, more generally μ_{aL} for an L-layer tissue structure) for that wavelength. The deep layer absorption coefficients at two wavelengths are then used to compute (block 1406) a deep layer SO₂ result, which is then output onto a user display 1408.

The lookup table 1370 is calibrated (i.e., loaded with precomputed numerical arrays) using the method of FIG. 13 separately for each distinct wavelength used, and will have functionally distinct numerical arrays stored for each different wavelength. Based on a population of physical tissue phantoms having known absorption coefficients, scattering coefficients, and layer thicknesses, a large population of calibration measurements 1312 is acquired using an aggregate NIR oximeter with multiple source-detector separation distances to produce values of an aggregate absorption coefficient μ_{aA} and aggregate scattering property μ_{sA} for each different separation distance D for tissue phantom. Alternatively, “virtual” tissue phantoms and computer simulations based thereon can be used in a manner similar to that of FIG. 2B, supra. Then, based on the measurements 1312 and known phantom parameters 1314, the lookup table 1370 is directly loaded. Preferably, the population of physical or virtual tissue phantoms is sufficient to span an entire range of realistic possibilities for the actual clinical values that will be encountered for the variables specified in blocks 1312 and 1314 of FIG. 13, such that direct readout and/or an interpolation algorithm internal to the lookup table circuitry will directly yield the desired readout of μ_{aBOT} (μ_{aL}) during clinical usage.

Use of a lookup table 1370 as the heart or “engine” of the deep-layer-specific NIR cerebral oximeter has been found advantageous in that results are computed very quickly. The use of lookup table 1370 has been further found particularly advantageous and practically achievable in view of the fact that the relationships of the involved parameters are generally “well-behaved” from a mathematical perspective, with functional relationships between (μ_{aTOP}, μ_{aBOT}, μ_{sTOP}, μ_{sBOT}, T, D1, D2 and (μ_{aA}, μ_{sA}) being generally smooth and continuously differentiable over their expected value ranges for any particular wavelength.

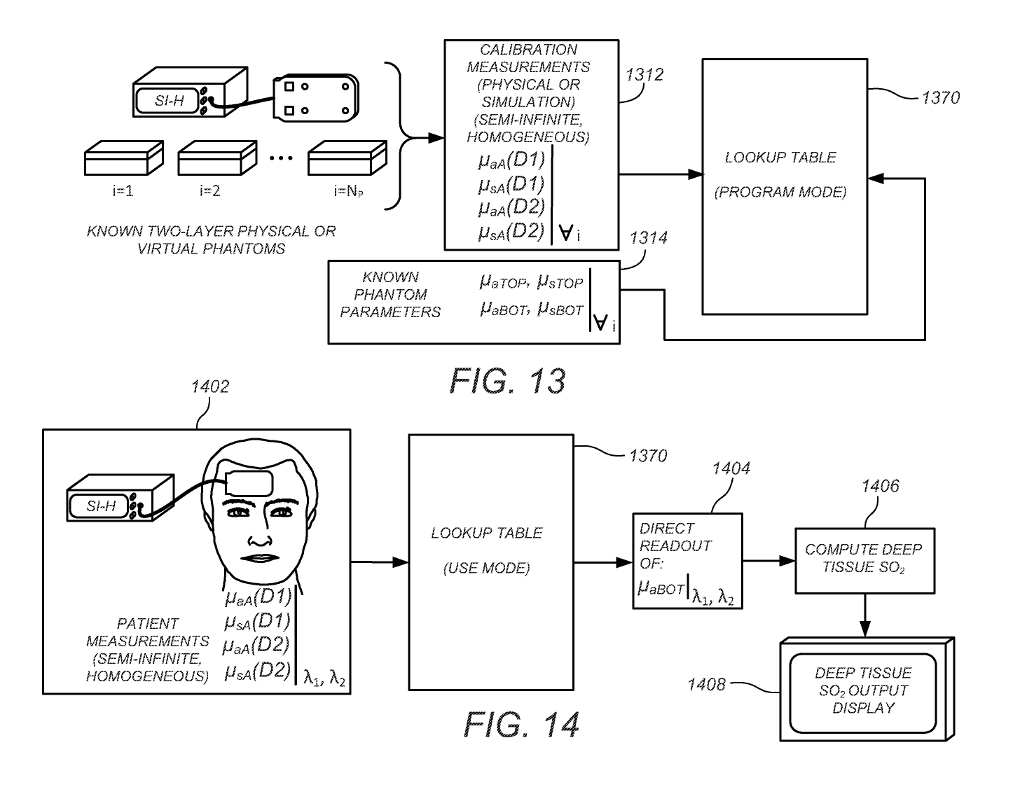

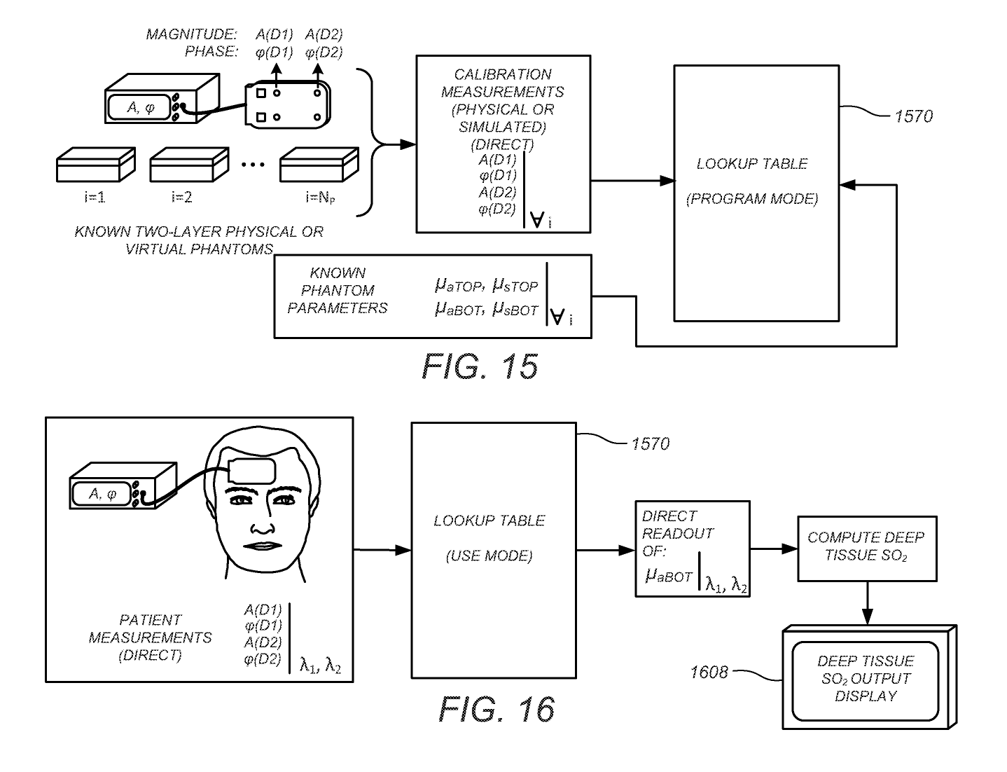

FIGS. 15-16 illustrate calibrating a deep-layer-specific NIR cerebral oximeter, and clinically using the calibrated deep-layer-specific NIR cerebral oximeter, respectively, according to a preferred embodiment in which a lookup table 1570 is used, except that direct amplitude and phase measurements A(D1), φ(D1), A(D2), φ(D2) corresponding to the multiple source-detector separation distances D1 and D2 are used as a basis for calibration programming and usage readout of the lookup tables. This represents a more direct approach than the preferred embodiment of FIGS. 13-14, in which absorption coefficients and scattering properties as computed under a semi-infinite, homogenous (SI-H) are used as the basis for programming and readout. For preferred embodiments in which multiple modulation frequencies are used at any particular wavelength (e.g., f1 and f2), multiple sets of the direct amplitude and phase measurements, that is, [A_{f1}(D1), φ_{f1}(D1), A_{f1}(D2), φ_{f1}(D2)] and [A_{f2}(D1), φ_{f2}(D1), A_{f2}(D2), φ_{f2}(D2)], will then be used in programming and reading out the lookup table for that particular wavelength. Notably, for the preferred embodiment of FIGS. 15-16, it is not required that the core NIR oximeter be programmed with an “aggregate” SI-H tissue property calculation algorithm, or any other tissue property calculation algorithm, because it is simply functioning to extract the output amplitudes and phases.

In another preferred embodiment (not shown) similar to that of FIGS. 15-16, the direct amplitude and phase measurements for both wavelengths (e.g. both wavelengths 690 nm and 830 nm) are used in conjunction with each other in programming and readout of the lookup table 1570, and the SO₂ reading is provided as a single, ultimate, direct readout of the lookup table 1570. For calibration purposes in this preferred embodiment, the “known” SO₂ reading for the bottom layer can be supplied to the lookup table 1570 in the program mode using the relationship SO_{2,BOT}=1.0941-0.4284{μ_{aBOT}(690)/μ_{aBOT}(830)}. In another preferred embodiment (not shown) similar to that of FIGS. 13-14, the SI-H (semi-infinite, homogenous assumption) calibration readouts for both wavelengths 690 nm and 830 nm are used in conjunction with each other in programming and readout of the lookup table 1370, and the SO₂ reading is provided as a single, ultimate, direct readout of the lookup table 1370.

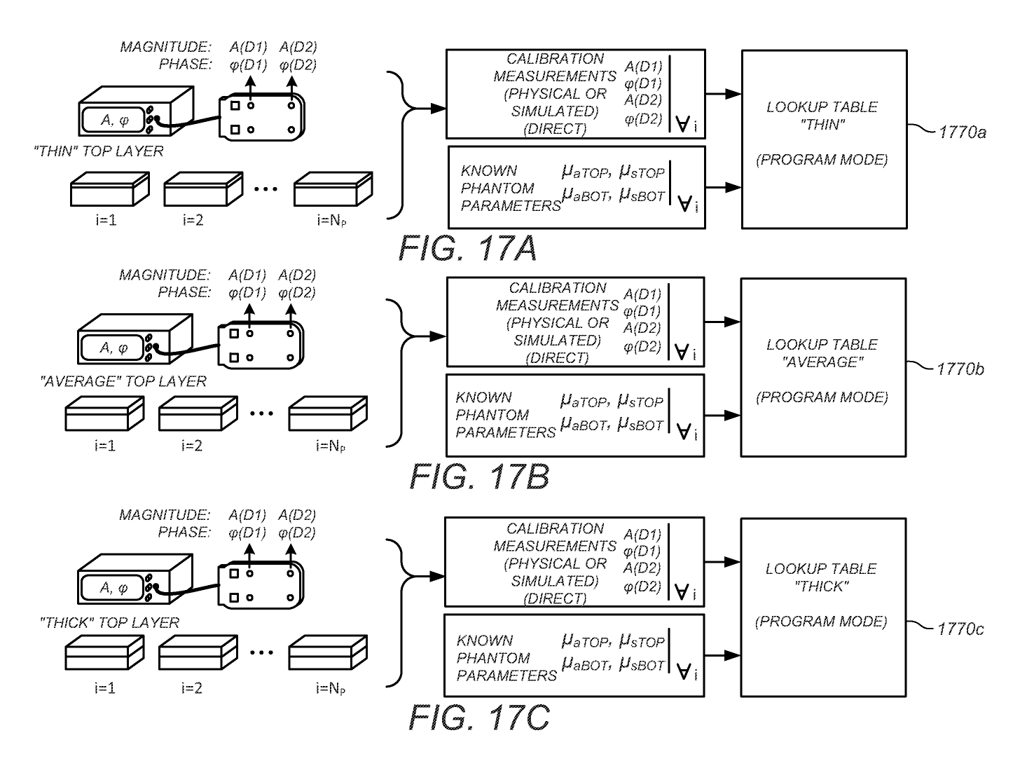

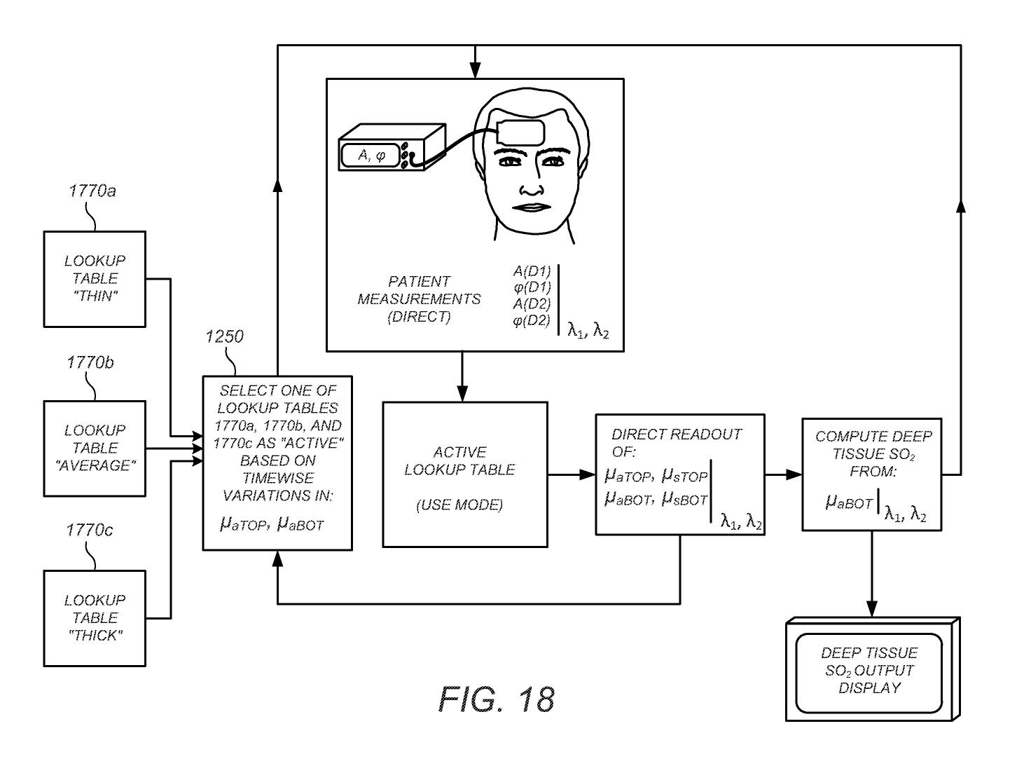

FIGS. 17A-17C and FIG. 18 illustrate multiple sets of dual-layer tissue phantoms (thin, average, thick top layer), corresponding calibrated lookup tables (1770a, 1770b, and 1770c), and forward usage of a corresponding lookup-table based deep-tissue-specific NIR cerebral oximeter, respectively, according to a preferred embodiment. The calibration process proceeds separately for each different top-layer thickness and results in separate lookup tables (or, equivalently, a single lookup table segregated into three parts). Forward usage of the calibrated lookup tables in FIG. 18 proceeds iteratively in a manner analogous to that shown in FIG. 12, supra, in selecting the proper lookup table (thin, average, thick top layer) except that the patient measurements 1202 are direct amplitudes and phases and a lookup table is used.

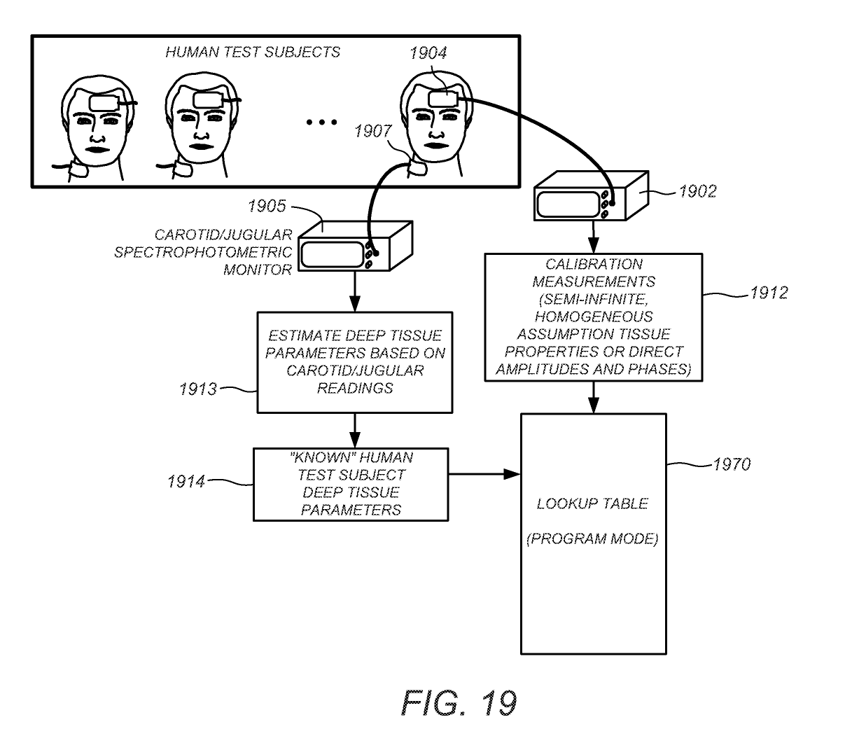

FIG. 19 illustrates calibrating a deep-layer-specific NIR cerebral oximeter according to a preferred embodiment in which a population of live human beings 1904 are used as calibration subjects. Using a separate, differently calibrated, non-invasive, “non-deep-tissue” NIR oximeter, such as a carotid/jugular NIR oximeter 1905 having a neck-mounted patch 1907, a deep tissue (brain tissue) parameter such as SO₂ is estimated (step 1913) is estimated based on acquired carotid/jugular readings of NIR oximeter 1905 for each human patient over a period of time. Concurrently with the readings of NIR oximeter 1905, an aggregate (core) NIR tissue oximeter 1902 (the same as will be used in forward usage mode on unknown patients) acquires calibration measurements 1912, which can either be absorption/scattering properties computed using a semi-infinite, homogeneous assumption or which can alternatively be direct amplitude and phase readings, across multiple source-detector separation distances, and preferably across multiple wavelengths. The estimations based on the readings of NIR oximeter 1905 are then used as the “known” brain tissue SO₂ readings 1914 for programming a lookup table 1970 in conjunction with the calibration measurements 1912.

Assumptions and methods used in converting the acquired carotid/jugular readings to “known” brain tissue SO₂ readings can be analogous to those described in Macleod. D. et. al., “Simultaneous Comparison of FORE-SIGHT and INVOS Cerebral Oximeters to Jugular Bulb and Arterial Co-Oximetry Measurements in Healthy Volunteers,” Anesth. Analg. 2009: 108 (SCA Suppl), which is incorporated by reference herein. As an optional alternative to the non-invasive NIR oximeter 1905 during the calibration process, blood samples drawn using a jugular bulb and a radial arterial catheter can be analyzed for oxygen tension using a blood gas co-oximeter, with estimates of “known” brain tissue SO₂ readings 1914 being derived therefrom. The calibrated lookup table 1970 can then be used in forward usage mode (not shown) in conjunction with the core NIR tissue oximeter 1902 to provide deep-layer-specific brain tissue SO₂ readings by direct lookup table readout.

The equations and algorithms disclosed in the instant specification can be implemented in hardware, software, or in a combination of hardware and software. The methods disclosed herein can be implemented by processing devices programmed to achieve the methods using known computer programming techniques. The programs can be designed to execute on programmable processors or computers, such as microcomputers, each including at least one processor, at least one data storage system (including volatile and non-volatile memory and/or storage elements), at least one input device, such as a keyboard or push button array, and at least one output device, such as an LCD display or printer. Program code is applied to input data to perform the functions described herein. The output information is applied to one or more output devices such as a printer, an LCD display (or other user display), or an internet-accessible web page as may be used for remote monitoring. Each program used in the systems described in the instant specification is preferably implemented in a high level procedural or object oriented programming language to communicate with a computer system. However, the programs can be implemented in assembly or machine language, if desired. The programming language can be, without limitation, a compiled or interpreted programming language. Each such computer program can be stored on a storage medium or device (e.g., ROM, hard disk drive, magnetic diskette) readable by a general or special purpose programmable computer, for configuring and operating the computer when the storage medium or device is read by the computer to perform the described procedures. The programs can also be considered to be implemented as a computer-readable storage medium, configured with a computer program, where the storage medium so configured causes a processor in the computer to operate in a specific and predefined manner to perform the described functions. Although any communications network can be used to obtain results from remote monitoring, the Internet or wireless systems represent useful choices for transmitting data.

Whereas many alterations and modifications of the present invention will no doubt become apparent to a person of ordinary skill in the art after having read the foregoing description, it is to be understood that the particular embodiments shown and described by way of illustration are in no way intended to be considered limiting. By way of example, in one or more of the preferred embodiments described above, the chromophore levels of the other “non-interesting” tissue layers may not be observable in conjunction with the deep-level-specific chromophore levels, especially for some preferred embodiments based on lookup-table generation in which it might not be necessary to use top-layer specific parameters or top-layer-specific calibration readings in order to sufficiently populate the lookup table(s). However, in other preferred embodiments, the chromophore levels of the other “non-interesting” tissue layers may indeed be observable, such during the progressive layer-by-layer computations performed in conjunction with the multilayer tissue models of FIGS. 7C-7D and FIGS. 10A-10B, supra. For such cases, it is within the scope of the preferred embodiments to simultaneously display the chromophore levels for all layers of the multilayer tissue structure, including both the “non-interesting” and “interesting” layers. Likewise, for those preferred embodiments supra in which the thicknesses of certain intervening tissue layers may be an object of computation (see, e.g., FIGS. 4-5) or iterative determination (see, e.g., FIG. 12 and FIG. 18), rather than simply being fixed thickness approximations, those computed or iteratively determined thicknesses can likewise be shown on the output display.

By way of further example, with reference generally to the preferred embodiments of FIGS. 1-12, supra, it is within the scope of the present teachings for the predetermined, pre-calibrated multi-layer tissue model of each example to be implemented as a neural network. With reference generally to the examples of FIGS. 13-19, it is within the scope of the present teachings for the lookup table of each example to be implemented as a neural network. Therefore, reference to the details of the embodiments are not intended to limit their scope, which is limited only by the scope of the claims set forth below.

What is claimed is:

1. A method for non-invasive spectrophotometric monitoring of a biological volume having multiple tissue layers including a relatively deep tissue layer, comprising:
receiving at least one measured absorption property and at least one measured scattering property for each of a plurality of predetermined source-detector separation distances of an aggregrate near-infrared spectrophotometric (NIRS) tissue monitor as applied along a surface of the biological volume, the aggregate NIRS tissue measurements being based on a model of the biological volume as a single-layer, semi-infinite, homogeneous volume;
receiving a predetermined multi-layer tissue model that characterizes a mathematical relationship among (a) absorption and scattering properties of each layer of a multi-layer tissue structure, and (b) aggregate absorption and scattering properties of the multi-layer tissue structure as would be measured by the aggregate NIRS tissue monitor at selected source-detector separation distances along a surface thereof; and
processing said measured absorption and scattering properties of said biological volume for said plurality of predetermined source-detector separation distances in conjunction with said predetermined multi-layer tissue model to compute therefrom a deep-layer-specific absorption property corresponding to the relatively deep tissue layer.

2. The method of claim 1, further comprising computing an oxygen saturation level for the relatively deep tissue layer based at least in part on said deep-layer-specific absorption property for the relatively deep tissue layer.

3. The method of claim 1, wherein said aggregate NIRS tissue monitor measures said absorption and scattering property using phase modulated spectrophotometry (PMS) for each of said source-detector separation distances.

4. The method of claim 1, wherein said predetermined multi-layer tissue model characterizes a mathematical relationship among (a) the absorption and scattering properties of each layer of the multi-layer tissue structure, (b) aggregate absorption and scattering properties of the multi-layer tissue structure as would be measured at a selected source-detector separation distance along a surface thereof, and (c) a thickness of each layer of the multi-layer tissue structure.

5. The method of claim 4, wherein said mathematical relationship comprises a representation of each aggregate absorption property and each aggregate scattering property as a Taylor series expansion of (i) the absorption property of each layer, (ii) the scattering property each layer (iii) the thickness of each layer, and (iv) the source-detector separation distance.

6. The method of claim 1, further comprising generating the predetermined multi-layer tissue model according to the steps of:
providing access to a population of multi-layer calibration volumes having known layer thicknesses and known absorption and scattering properties in each layer;
acquiring, for each said multi-layer calibration volume, aggregate absorption and scattering properties as would be measured at selected source-detector separation distances along a surface thereof; and
processing the acquired aggregate absorption and scattering properties in conjunction with said known layer thicknesses and said known absorption and scattering properties of each layer to generate said multi-layer tissue model.

7. The method of claim 6, wherein each said multi-layer calibration volume is a physical multi-layer tissue phantom, and wherein said acquiring said aggregate absorption and scattering properties comprises applying said aggregrate NIRS tissue monitor to each said physical multi-layer tissue phantom at multiple source-detector separation distances.

8. The method of claim 6, wherein each said multi-layer calibration volume is a virtual multi-layer tissue phantom, and wherein said acquiring said aggregate absorption and scattering properties comprises applying a NIRS computer simulation algorithm to each said virtual multi-layer tissue phantom for multiple source-detector separation distances.

9. The method of claim 1, wherein said predetermined multi-layer tissue model is expressed in the form of a lookup table, and wherein said processing said measured absorption and scattering properties comprises applying said measured absorption and scattering properties to said lookup table.

10. The method of claim 1, wherein said biological volume is the human head, and wherein said predetermined multi-layer tissue model is a two-layer model including (i) a shallow layer jointly representative of the skin, skull, and cerebrospinal fluid layers, and (ii) a deep layer representative of the brain.

11. An apparatus for non-invasive spectrophotometric monitoring of a biological volume having multiple tissue layers including a relatively deep tissue layer, comprising:
an aggregrate near-infrared spectrophotometric (NIRS) tissue monitor configured to measure at least one aggregate absorption property and at least one aggregate scattering property for each of a plurality of predetermined source-detector separation distances along a surface of the biological volume, wherein the aggregate NIRS monitor computes the said absorption and scattering properties based on a model of the biological volume as a single-layer, semi-infinite, homogeneous volume;
a processing device programmed and configured to carry out the steps of:
receiving the at least one aggregate absorption property and the at least one aggregate scattering property for each of the plurality of predetermined source-detector separation distances from the aggregate NIRS tissue monitor;
receiving a predetermined multi-layer tissue model that characterizes a mathematical relationship among (a) absorption and scattering properties of each layer of a multi-layer tissue structure, and (b) aggregate absorption and scattering properties of the multi-layer tissue structure as would be measured by the aggregate NIRS tissue monitor at selected source-detector separation distances along a surface thereof; and
processing said measured absorption and scattering properties of said biological volume for said plurality of predetermined source-detector separation distances in conjunction with said predetermined multi-layer tissue model to compute therefrom a deep-layer-specific absorption property corresponding to the relatively deep tissue layer;
and a display device coupled to the processing device for outputting a display of at least one deep-layer-specific biological metric computed at least in part from said deep-layer-specific absorption property.

12. The apparatus of claim 11, wherein said at least one deep-layer-specific biological metric includes a tissue oxygen saturation metric, and wherein said aggregate NIRS tissue monitor measures said absorption and scattering property using phase modulated spectrophotometry (PMS) for each of said source-detector separation distances.

13. The apparatus of claim 11, wherein said predetermined multi-layer tissue model characterizes a mathematical relationship among (a) the absorption and scattering properties of each layer of the multi-layer tissue structure, (b) aggregate absorption and scattering properties of the multi-layer tissue structure as would be measured at a selected source-detector separation distance along a surface thereof, and (c) a thickness of each layer of the multi-layer tissue structure.

14. The apparatus of claim 11, wherein said predetermined multi-layer tissue model is expressed in the form of a lookup table, and wherein said processing said measured absorption and scattering properties comprises applying said measured absorption and scattering properties to said lookup table.

15. The apparatus of claim 11, wherein said biological volume is the human head, and wherein said predetermined multi-layer tissue model is a two-layer model including (i) a shallow layer jointly representative of the skin, skull, and cerebrospinal fluid layers, and (ii) a deep layer representative of the brain.

16. The apparatus of claim 11, further comprising a calibration processor configured and programmed to carry out the steps of:
receiving, for each of a population of multi-layer calibration volumes having known layer thicknesses and known absorption and scattering properties in each layer, aggregate absorption and scattering properties as would be measured at selected source-detector separation distances along a surface thereof; and
processing the acquired aggregate absorption and scattering properties in conjunction with said known layer thicknesses and said known absorption and scattering properties of each layer to generate said multi-layer tissue model.

17. The apparatus of claim 16, wherein each said multi-layer calibration volume is a physical multi-layer tissue phantom, and wherein said aggregate absorption and scattering properties received by the calibration processor are acquired by application of said aggregrate NIRS tissue monitor to each said physical multi-layer tissue phantom.

18. A computer program product embodied on a computer-readable medium for non-invasive spectrophotometric monitoring of a biological volume having multiple tissue layers including a relatively deep tissue layer, comprising:
computer code for receiving at least one measured absorption property and at least one measured scattering property for each of a plurality of predetermined source-detector separation distances of an aggregrate near-infrared spectrophotometric (NIRS) tissue monitor as applied along a surface of the biological volume, the aggregate NIRS tissue measurements being based on a model of the biological volume as a single-layer, semi-infinite, homogeneous volume;
computer code for receiving a predetermined multi-layer tissue model that characterizes a mathematical relationship among (a) absorption and scattering properties of each layer of a multi-layer tissue structure, and (b) aggregate absorption and scattering properties of the multi-layer tissue structure as would be measured by the aggregate NIRS tissue monitor at selected source-detector separation distances along a surface thereof; and
computer code for processing said measured absorption and scattering properties of said biological volume for said plurality of predetermined source-detector separation distances in conjunction with said predetermined multi-layer tissue model to compute therefrom a deep-layer-specific absorption property corresponding to the relatively deep tissue layer.

19. The computer program product of claim 18, wherein said biological volume is the human head, and wherein said predetermined multi-layer tissue model is a two-layer model including (i) a shallow layer jointly representative of the skin, skull, and cerebrospinal fluid layers, and (ii) a deep layer representative of the brain.

20. The computer program product of claim 18, wherein said predetermined multi-layer tissue model is expressed in the form of a lookup table, wherein said computer code for processing said measured absorption and scattering properties comprises computer code for applying said measured absorption and scattering properties to said lookup table, and wherein said computer program product further comprises computer code for generating a deep-layer-specific tissue oxygen saturation metric based at least in part on said deep-layer-specific absorption property.
